(19) United States
(12) Patent Application Publication
(10) Pub. No.: US 2006/0057555 A1
(43) Pub. Date: Mar. 16, 2006

(54) Method and system for controlling the development of biological entities

(76) Inventors: Udi Damari, Ganiey Tikva (IL); Amir Shaham, Yavneh (IL); Ginadi Shaham, Yavneh (IL); Uri Meir, Kibbutz Bet Hashita (IL)

Correspondence Address: NATH & ASSOCIATES, 112 South West Street, Alexandria, VA, 22314, US

(21) Appl. No.: 10/519,093

(22) PCT Filed: Jun. 29, 2003
(86) PCT No.: PCT/IL03/00544

§ 371 Date: Apr. 11, 2005

Related U.S. Application Data
(60) Provisional application No. 60/391,575, filed on Jun. 27, 2002.
(60) Provisional application No. 60/417,460, filed on Oct. 10, 2002.

(51) Int. Cl. C12Q 1/00; G06F 19/00

(52) U.S. Cl. 435/4; 702/19

(57) ABSTRACT

A method and system are presented for use in controlling the processing of components, e.g. biological entities. Each component is assigned with a unique machine readable identification mark. Data records are provided representative of matching sets of the identification marks relating to at least two associated components. Each component-containing holder may be provided with the unique machine readable identification mark assigned to the biological entity in the holder. This enables to identify whether the biological entities to be processed relate to the matching set or not.

### FIELD OF THE INVENTION

This invention relates in general to monitoring and control of biological entities (e.g. cells) during their development as well as to the preservation of biological entities. The invention is particularly useful for monitoring and controlling the entities' development during incubation and/or with Assisted Reproductive Technologies (ART)

### BACKGROUND OF THE INVENTION

In the fields of cyropreservation and reproduction of biological entities many techniques and supporting technologies, including microscope monitoring, have been and still are under development. A microscope is the basic tool for most biological procedures. In every laboratory there are usually several types of microscopes and most of these devices have a special adapter for video or CCD camera.

One improvement of the basic imaging technology is described in U.S. Pat. No. 6,166,761. This technique allows for reducing the need for a microscope, and utilizes very small CCDs or video cameras with special adapters and microscopic lenses to produce high quality imaging of biological entities in an incubator.

Embryo development is one example of a biological procedure to be monitored and controlled during ART, by the use of the different cyropreservation and reproduction technologies under development.

Infertility problems and treatment thereof is a growing area of healthcare. Many approaches are being developed to resolve problems with infertility. Infertility is understood to be the inability to conceive, within a certain period of time from sexual intercourse, without the use of contraceptives. Because infertility exerts extreme physical, emotional and financial stresses on those who are unable to conceive, there is a great need for improved aids for reproduction. One common treatment is In Vitro Fertilization (IVF), which has grown explosively in the two decades since it was developed. In its simplest form, IVF consists of pharmaceutical stimulation of the female's ovaries to produce a large number of eggs. Eggs surgically harvested from these follicles are then mixed in the laboratory with the male's sperm. If fertilization is successful, the embryos are incubated for a short time and then transferred back to the female. If one of these embryos implants in the uterine wall, a successful pregnancy may follow.

There are several modifications of the basic IVF technique. For example, intracytoplasmic sperm injection (ICSI) can be used for cases of low sperm count or cases where the sperm has difficulty fertilizing the egg. Another IVF modification is Assisted Hatching (AH), a procedure in which the zona pellucida (the outer wall of the embryo) is mechanically cut or chemically etched, thereby partially exposing the embryo. In some laboratories, this procedure significantly improves implantation rates, particularly for older patients. Finally, IVF procedures can also incorporate donor tissues, including sperm, ova and embryos, for those individuals who cannot produce their own.

Despite its great successes, IVF has several significant problems. First and foremost, the procedure is unpredictable. Although the ideal result of any IVF procedure is a single, live birth, a viable pregnancy occurs in only about 30% of all procedures. Conversely, IVF may result in a pregnancy with multiple births, because in most cases, physicians implant more than one embryo into the patient, in order to increase the chance for a successful pregnancy. In this regard, twins and triplets pose high risk on the babies as well as on the mother, comparing to a single embryo pregnancy. The potential for problems, however, increases for higher order births. Selective embryo reduction is therefore often recommended for these cases.

IVF begins with a source of sperm and oocyte, using donor tissues if necessary. Next, fertilization occurs, and good IVF laboratories typically have a fertilization success rate of about 75%, using ICSI if appropriate. After a short incubation period, the resulting embryo is then introduced into the uterus, where implantation occurs.

Implantation is generally the limiting factor in overall IVF success. The implantation process is actually quite complicated and requires the coordination of many factors, some of which are unknown. A failure of any one of these processes prevents implantation and thus pregnancy.

Observing embryos during early stage development is one of the best means for selecting the best embryo for transfer and accordingly of enhancing the chances of successful implantation. The better chance of success would allow the transfer of only a single embryo, and thus avoiding the risk of multiple births.

Presently, the monitoring of embryos in vitro is conducted manually, by embryologists. The embryos are taken out of the incubator, and evaluated several times a day under a stereo-microscope located outside the incubator. However, this approach has several limitations that may result in damage to the embryos. This method is detrimental for the preservation of optimal conditions necessary for embryo development and causes fluctuations of temperature from the optimal value (typically 37° C. or preferably 36.7° C.), uncontrolled CO₂ environment and light exposure for approximately 10 minutes a day. In addition, when higher magnification is necessary the embryos are observed with an inverted microscope, which is normally located outside the laminar flow which increases the risk for contamination.

In addition, during ART procedures special attention is paid to the issue of matching between oocytes, sperms and patient or embryos and patient. Even a minor mistake could lead to a personal disaster for the future parents. Only recently, an IVF mix-up occurred where black twins were born to a white couple (http://news.bbc.co.uk/1/hi/health/211552.stm).

### SUMMARY OF THE INVENTION

There is a need in the art to facilitate monitoring and controlling of biological entities development by providing a novel optical method and system.

The present invention provides for monitoring the development of embryos while remaining in a stable controlled environment, and thus assuring the best conditions for embryo development. The present invention also provides for carefully monitoring the processing of the components involved in the ART procedure, including identifying the holders and containers of the biological entities (including oocyte, ovary slices, sperm and/or embryo), the patients and the databases, files and records involved in the ART procedure during the entire ART procedures, thus enabling to avoid blunders.

The method and system of the present invention provides for the monitoring, including continuous monitoring of entities, in particular, embryos, during their development in a controlled environment (or incubator), without the need to take the entity out of this environment for the purpose of monitoring. The technique of the present invention thus enables to continuously retain the biological entity, while being monitored, under a stable controlled environment.

The incubator is known as closed environment for maintaining biological entities, which closed environment is equipped with the standard environment control means, such as temperature and humidity control means, CO₂ and oxygen levels' control means, aseptic environment control means, etc. One example of an incubator which may be utilized in the present invention is described in U.S. Pat. No. 6,166,761,assigned to the assignee of the present application. This incubator includes two or more mini-CCDs operable as mini-photomicroscopes. The entire system can be configured for time-lapse photomicroscopy, transmission photomicroscopy, reflection photomicroscopy, epifluorescence photomicroscopy, or infrared photomicroscopy. 3D images can be acquired by focusing the mini-photomicroscopes on successive focal planes in the biological entities. The mini-photomicroscopes may be focused on separate entities, on different portions of the same entity, or on the same portion of the same entity.

For the purpose of this application, the terms “incubator” or “controlled environment” define an environment, preferably closed or substantially closed, which has at least a temperature control means and may also have humidity, CO₂ and oxygen levels' control means, aseptic environment control means, etc. and may be used for maintenance, preservation or development of biological entities. The present invention, in some of its aspects, may be used in an incubator, or may comprise an incubator but may also include use of components situated outside an incubator.

As appreciated, taking a biological entity out of the incubator may harm the entity or its development. At times, there is a need to monitor a biological entity or its development (e.g., for example at predefined time-lapse during the development process, e.g. every three hours, such as in the case of embryo development, which greatly increases the risk of harming the biological sample. Yet further, using a microscope for monitoring the biological entity outside the incubator requires that the biological entity be contained inside a special solution (under oil), which may damage the sample.

The present invention provides, in some of its aspects, for ensuring matching between at least two components that are to be involved in a common processing. Such components may include biological entities, including biological entities that comprise for example sperm, oocytes, embryo, blood, bone marrow, bacteria/antibiotic, drug or other agents, biological entity holders or containers, databases, files or records and also humans or animals serving as donor subjects, recipients, physicians, technicians etc. To this end, the present invention utilizes assigning each component with a unique machine readable identification mark (e.g., barcode), which is associated with this component or a specific set of components. The mark is identifiable through appropriate processing, for example image processing.

When a specific set of components is assigned with the same unique identification code or matching identification codes, the invention provides for concurrent identification of the components to enable matching therebetween. The invention thereby practically prevents mix-up of components or erroneous manipulation of components belonging to different matching sets. Examples for such mix-ups may be the fertilization of an egg with non-matching sperm; transfer of an embryo to a wrong recipient, use of data or instructions from a file relating to one patient in respect of another unrelated patient, transfer of a biological entity to a holder that is not form the matching set of the entity, etc.).

The present invention provides a system and method for monitoring a biological entity or a process such as the embryo development process. This aspect is referred to hereinafter as “the embryo development embodiment”. According to this embodiment, each embryo is automatically monitored at predefined time-lapse during the embryo development (e.g., every three hours), data indicative of the embryo development conditions are stored, for example, as image files. It should be appreciated that the image files may be in the form of a single frame image or in the form of continuous frames (e.g. a short video film). Each entity has its unique record (referred to as the “embryo record”). The system of the present invention may utilize a modified incubator additionally equipped with optical, robotic and control means, or may comprise a special incubator constructed as part of the system. The optical means includes at least one image acquisition system utilizing magnification optics (e.g., microscopic lens arrangement). The control means includes an external control system that is typically a computer system installed with specific utilities (hardware and/or software) and may utilize an image processing technique which may also be based on pattern recognition. The robotic means is operable by the control means and includes an entity or entity holder or container positioning assembly for locating a selected one of the entities or holders within an imaging plane. The system may also comprise a catheter/pipette for inserting required solutions into the entity drops to thereby control the media environment thereof. The system may comprise a laser-assisted etcher for assisted hatching of the embryo. The system may also comprise a syringe and robotic means for moving such syringe for performing ICSI procedure, meaning to inject a single sperm cell into an oocytes/egg.

The system may also have elements that should minimize the probability of taking an undesired entity out of the incubator. This is achieved by placing an identifying mark on the entity or the entity holder or container identification mark and providing means for allowing the user to take out only the requested entity or entity holder. Such means can be provided by a mechanical element, such as barrier, or by positioning the entity or the holder in a specific position ready for dispatching out.

The present invention provides a system and method for controlling a process of biological entities matching. This is implemented by using a holder for one or more of the biological entities each labeled with a unique identification code assigned to the respective biological entity, and a matcher device. The latter has a support platform for supporting the labeled holders, and an imaging arrangement operable to acquire images of the labels and generate data indicative thereof. The previously created records representative of matching sets of biological entities' identification codes are used for analyzing the image generated data to identify the identification codes and determine whether the respective biological entities belong to a matching set.

Thus according to one broad aspect of the present invention, there is provided a method for use in controlling the processing of components the method comprising:
assigning each component with a unique machine readable identification mark;
providing data records representative of matching sets of the identification marks relating to at least two associated components; and
providing on each component the unique machine readable identification mark assigned to said component, thereby enabling to identify whether the components to be processed relate to the matching set or not.

It should be understood that the term “processing” used herein signifies also monitoring of the biological entity condition, but may also signify a biological procedure such as IVF or a documentation process or a mixture of the above.

According to another broad aspect of the present invention, there is provided a system for use in controlling the processing of biological entities, the system comprising:
a support assembly for supporting at least one holder containing biological entities;
an optical device operable to acquire an image of the holder and generating data indicative of at least an identification mark provided on the holder;
a control system connectable to said optical system and operable to actuate the image acquisition and to analyze the data indicative of the acquired images, the control system having a memory utility for storing reference data representative of matching sets of biological entities' associated identification marks, and a processing utility preprogrammed to be response of said data indicative of the acquired images to analyze said data utilizing said reference data and identify whether the biological entities in at least two holders relate to a matching set or not.

According to yet another broad aspect of the present invention, there is provided a label for attaching to a biological entity holder, said label comprising a machine readable identification mark assigned to a biological entity to be put in said holder and a pattern defining a plurality of spaced-apart sites for a plurality of the biological entity drops, respectively.

The present invention according to its yet another aspect provides a holder for a specific biological entity, the holder carrying a machine readable identification mark assigned to said biological entity.

According to yet further aspect of the invention, there is provided a cover arrangement to be used for covering a surface of a support stage for supporting biological entity containing holders in spaced-apart relationship, the cover being configured to cover the entire surface region intended for location of the biological entity containing holders, and being formed with a recess that has a size of about that of the holder and substantially not exceeding that of the space between the holders, relative displacement between the cover and the biological entity holders' supporting surface thereby enabling to bring a selected one of the biological entities' holders to be located within said recess in the cover.

The present invention also provides a support stage for use to support a biological entity containing holder, the support stage comprising a temperature control arrangement mounted in the stage below a surface of the stage intended for supporting the holder, said temperature control arrangement comprising a heat sink unit for fluid, for example gas (e.g. air) or liquid (e.g. water), circulation therethrough, said heat sink unit including at least one temperature sensor and being controllably connectable to an external heat sink unit to enable selective supply of fluid to said heat sink unit inside the support stage upon detecting that the temperature condition of the stage has changed.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIGS. 1A to 1C illustrate biological entities' holders according to the invention, wherein FIG. 1A illustrates an example of a dish holder, FIG. 1B illustrates and example of a tube like holder and FIG. 1C depicts an example for a physical barrier within a holder for limiting the embryo displacement within the respective site;

FIG. 2A is a schematic illustration of a matcher device according to the invention;

FIG. 2B is a schematic illustration of a support assembly according to the invention for supporting a tube-like holder and that us useful in an assembly a matcher device;

FIG. 3 is a schematic illustration of an incubator equipped with a monitoring system according to the invention;

FIG. 4 schematically illustrates one example of the implementation of the imaging system useful in a monitoring system according to the invention;

FIG. 5 is a schematic illustration of a medium changer assembly for changing medium in an entity drop and/or maintaining controlled environment within the entity drop;

FIG. 6 exemplifies the evaluation scheme, which is obtained while monitoring embryos while in an incubator with the monitoring system according to the invention, and is useful for selecting the best embryo for implantation;

FIG. 7 exemplifies a temperature control system according to the invention suitable to be used in the monitoring system;

FIGS. 8A and 8B schematically illustrate side and top views of a cover according to the invention useful to reduce environmental changes experienced by the biological entities when an incubator is opened;

FIG. 9A schematically illustrates the main functional elements and main operational steps according to the invention for monitoring and controlling an IVF process;

FIG. 9B schematically illustrates how the matcher device according to the invention can be used for controlling various steps of the IVF or ICSI procedures; and

FIGS. 10 to 13 illustrate the results of an experiment conducted by the inventor to prove that the technique of the present invention bears no significant influence on the development of embryos in a culture dish, wherein FIG. 10 compares the results obtained for two groups of control entities (embryos)-control 1 (kept in an incubator that also contains the monitoring system of the invention) and control 2 (kept in the conventional incubator); FIG. 11 compares control 1 group to a group of embryos monitored by the system of the invention; FIG. 12 compares control 2 group to a group of embryos with an identification label (sticker) according to the invention being attached to the petri dishes; and

FIG. 13 is a graph showing the temperature measured in the system of the present invention as a function of time.

### DETAILED DESCRIPTION OF THE INVENTION

More specifically, the present invention is useful for monitoring and controlling of ART (such as IVF) and is therefore described below with respect to this specific application. It should, however, be understood that the invention may be useful for other applications dealing with biological entities monitoring or handling.

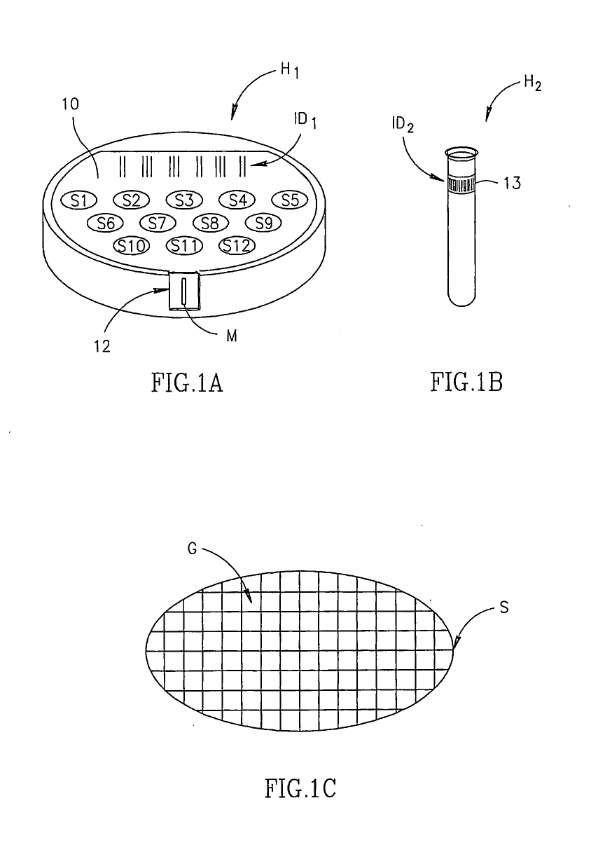

Referring to FIGS. 1A and 1B, there are illustrated two holders H₁ and H₂ according to the invention for containing a matching pair of oocyte and sperm entities, respectively. Here, the term “matching entities” signifies that these entities are to be involved in a common fertilization process. Considering a general biological procedure, there may be a plurality of entities relating to a specific set of entities, called “matching entities”. For example, the matching entities in the present example would also comprise the intended recipient of the embryos that would result from fertilization of said oocytes with said sperm, and a specific physician or technician involved in the procedure, and data records.

The holder H₁ of FIG. 1A is shaped like a dish or plate for carrying oocytes. The construction of such a dish is generally known and therefore needs not be more specifically described. A common example of such a dish is a petri dish. According to the invention, the dish H₁ at its outer surface is provided with a label 10 that includes a unique identification mark ID₁ assigned for example to the specific female, to whom the specific oocyte entities belong, and has a pattern defining a certain number of spaced-apart sites—12 such sites S₁-S₁₂ in the present example, each for carrying one of the entity drop containing a single oocyte (although it can contain more than one oocyte). The sites S₁-S₁₂ are assigned with identification marks (numbers) 1, . . . , 12. Such a pattern may be in the form of a plurality of spaced-apart holes. Generally, at least the pattern elements (sites) of the label 10, and preferably, but not necessarily, the entire label region on which the ID₁ is printed, are transparent, or partly transparent, with respect to predetermined radiation. The holder H₁ is preferably formed with a projecting portion 12 that serves for assisting in attaching the label to the dish surface. The portion 12 is preferably formed with a marker M (black line) for assisting alignment of the dish-with-label during further procedures. Preferably, in order to facilitate imaging of the biological entity within each site S of the dish, a substantially fixed position of the cell (or embryo) in the site is provided. This can be implemented by using any of the known methods, such as providing a suitable biological glue within the medium containing the cell, or by providing a physical barrier limiting the embryo displacement within the respective site, as described below with reference to FIG. 1c.

The holder H₂ shown in FIG. 1B is preferably shaped like a tube, and is also attached with a label 13 including an identification code ID₂, which may and may not be identical to ID₁, but is that relating to the ID₁ namely recorded as matching with ID₁. In other words, the codes ID₁ and ID₂ present a matching set (matching pair in this specific example).

It should be noted that the identification mark ID, may be of any suitable kind, preferably of a computer readable kind, such as image or scan readable (e.g., barcode as exemplified in FIG. 1), RF tag, magnetic tag, etc. These tagging means, as well as their realization and reading, are known per se and therefore need not be specifically described, except to note that barcode for example can be read using variety of technologies, including imaging the entire code or by scanning the code (e.g., by IR detector, UV beams, etc.).

It should also be noted that the ID₁ or ID₂ may be printed directly on the holder. If the use of ID-containing label is considered, the label may be attached to the holder by electrostatic means, by using organic glues (such as fibrinogen derived materials) or any other means, which do not influence or damage the entity in the holder.

The provision of such matching IDs on the pair of holders of biological entities to be paired, allows for automatic authentication of two entity-containing holders arriving to a fertilization stage.

As shown in FIG. 1C, the holder H₁ can be also preferably designed so as to facilitate imaging of the cell (embryo) in each of the cell sites S, namely, to enable fixing the cell position within the respective site. This can be implemented by making the inner surface of the holder within the site S with a certain surface relief defining groove(s) or by providing a mesh (grid) G in the site preventing, or limiting, the embryo's displacement within the site.

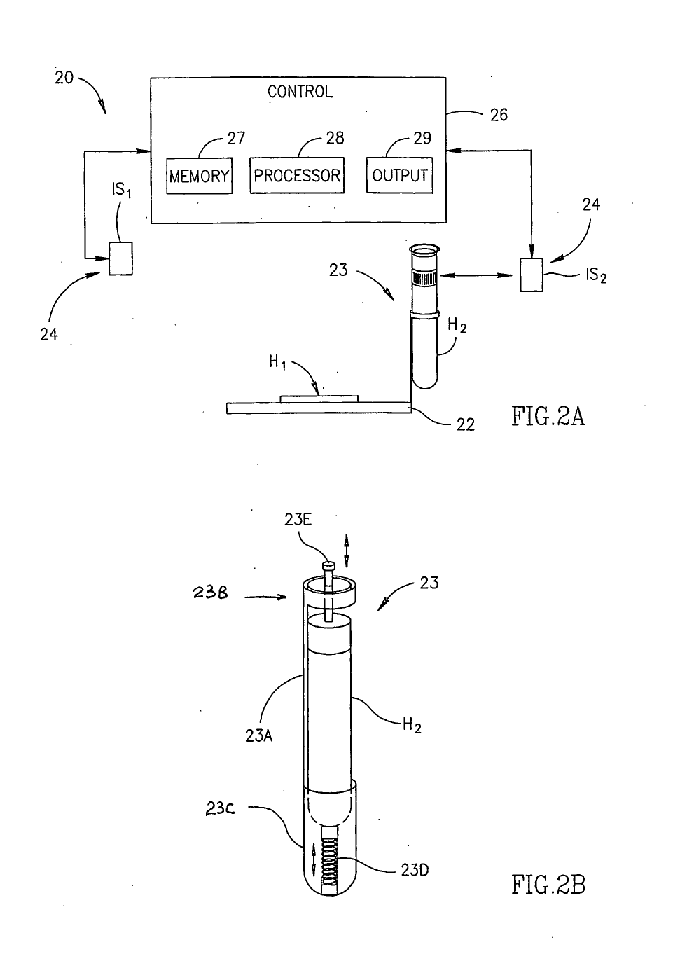

FIG. 2A illustrates a matcher device 20 according to the invention. The matcher device is an ID-controlled system (e.g., barcode-controlled) developed to prevent mismatching during IVF procedures. The matcher device can be used as part of, or in association with, the entire monitoring system of the present invention (which can be partly or completely installed within an incubator or can have incubation elements) as will be described further below, or as a stand-alone unit.

The matcher device can serve as an essential tool in the quality control process of the IVF procedure (or any other suitable procedure) and enables linking a number of such matcher devices to one central computer system, or any other data processing and gathering system.

The matcher device 20 comprises a substantially flat platform 22 for supporting the holder H₁, a support assembly 23 for supporting the holder H₂, an ID reading assembly 24, and a control system 26. The control system 26 is connectable to the ID reading assembly 24 (through wires or wireless) and connectable to a central station (not shown). It should be noted although not specifically shown here that the platform 22 is preferably equipped with temperature and/or gas (e.g., CO₂) control means. The support assembly 23 is preferably attachable to the platform 22, and may generally be of any suitable design capable of carrying a tube-like holder in a substantially fixed position.

FIG. 2B exemplifies the construction of the support assembly 23 according to the invention aimed at enabling to support in a substantially fixed manner a holder (tube) H₂ of any possible height. To this end, the support assembly 23 includes an elongated member 23A having spaced-apart top and bottom elements 23B and 23C defining a space therebetween for locating the tube H₂. Mounted in these top and bottom elements are spring and screw assemblies 23D and 23E, respectively. When the tube H₂ is installed in the support assembly 23, the position of the screw 23E is adjusted to engage the top portion of the tube, while the bottom end of the tube is kept against the tension of the spring 23D. A series of assemblies such as that exemplified in FIG. 2B facilitates the simultaneous reading of the IDs of several holders of different dimensions by allowing the adjustment of their positions such that all these ID's will be in the same focal plane.

Turning back to FIG. 2A, in the present example, the ID reading assembly 24 is an optical assembly, and comprises two imaging systems IS₁ and IS₂ associated with the holders H₁ and H₂, respectively, for capturing images of the biological entities in these holders. These imaging systems utilize a common or separate light sources generating radiation of a spectral range and exposure time causing no damage to biological entities (such damage typically including peroxidation of the cells' lipids)(typically excluding UV and IR). The entire imaging system IS₁ or at least its light source may be located below the dish (the so-called “back illumination” mode), in which case the platform would be transparent or would be of a frame-like design allowing back illuminated light propagation to the dish H₁. Each of the imaging systems IS₁ and IS₂ comprises its own lens arrangement and optical detector. Such an imaging system may be a digital or video camera.

It should be noted that, generally, a single imaging system (digital or video camera) could be used for the same purposes, namely for imaging more than one holder. In this case, the imaging system is operated by the control unit 26 to sequentially acquire images of the labeled holders (generally, ID-marked holders). The holder supporting assembly and/or the optics may be appropriately displaceable one with respect to the other to provide the desired position of the entity in holder for imaging purposes. When using separate imaging systems for imaging the separate holders, image acquisition can advantageously be carried out substantially simultaneously. It should also be noted that for reading the corresponding identification codes attached to the patients (or hard copy files) involved in this specific IVF procedure (e.g., ID printed on bracelet carried by the patient or printed on a label attached to the bracelet), a portable ID detector can be used, for example a barcode scanner.

The control unit 26 is a computer system including inter alia a memory utility 27 for storing reference data representative of the matching ID sets previously recorded; a processor utility 28; and a data output utility 29 (e.g., display, or audio or light indicator). The processor utility 28 is preprogrammed with suitable software for analyzing data indicative of the IDs read by the assembly 24 (e.g., by carrying out pattern recognition based image processing) while utilizing the reference data stored in the memory utility, to determine whether these IDs match each other or not, and generate a signal indicative thereof to operate the output utility 29. The control unit preferably also comprises suitable data input means (not shown) to enable input relevant data, for example the name of a physician responsible for this procedure. The matcher device is preferably portable, having a display (preferably an LCD monitor), and a small data input means (e.g., keyboard or touch screen or any other suitable type of button(s)) to conduct command operations, for example from the central computer station.

The control unit 26 thus operates the imaging systems IS₁ and IS₂ and possibly also operates the holders' support assemblies to enable substantially simultaneously acquire images of the IDs on the two holders (or operates the single imaging system to enable sequential image acquisition), and then operates to analyze data indicative of the acquired images and/or the identification marks, or operates to transfer data to a central computer station.

Based upon the characteristics of the designated action to be performed, the matcher device of the present invention operates a generic testing procedure that includes the number of components it is meant to test, and to verify the correctness of the code. The entire operative process is conducted in the holder, as long as it is still located outside the mother instrument, is accompanied by reviewing between the holder H₁ (e.g. petri dish or other such laboratory dishe/container) and the holder H₂ (e.g. test tube or cryogenic vial or other such laboratory containers). In the event that at some point a discrepancy should occur in the content of the ID (e.g., barcode), the processor utility 28 alerts of the same by sending a notice to the data output utility 29, which in turn may transmit a corresponding record of the discrepancy to the central station (computer system or any other suitable data processing means), operation of a buzzer and/or lighting up a flashing red LED (or any other suitable indicative colored light means) until the problem is solved by the user. The central computer system conducts a complete recording of all the ID comparative actions from all the system units.

It should also be noted that, generally, the reference data indicative of matching IDs may be stored in the memory utility of the central station, and the matcher device communicates with the central station via wire-based or wireless signal trasmission.

The Petri dishes H₁ are preferably placed at the ID-testing phase in the matcher device upon the platform 22 is heated to a required temperature (about 37° C., typically 36.7° C.) under control of a microprocessor and temperature sensor (or any other temperature control means). The temperature condition is permanently monitored (e.g., shown on the screen). The sperm vials H₂ are located in an unheated surface area. The matcher device preferably includes backlight surfaces based on the White LED technology (or any other illuminating technology undamaging for a biological entity). The dish and vial charging surface is located opposite optical sensors. This indicates to the system if they are present or absent from the waiting position.

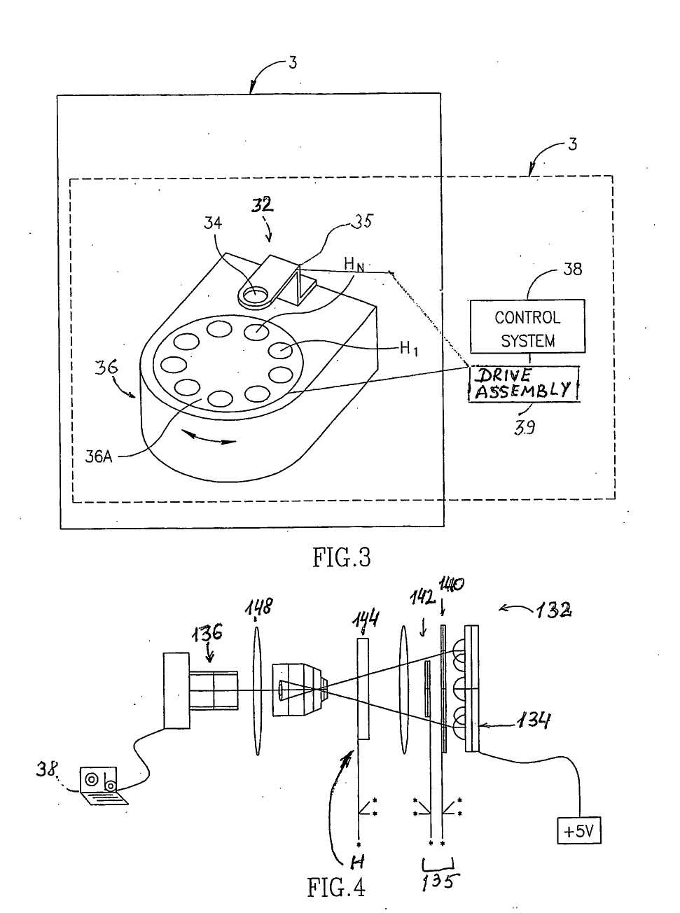

FIG. 3 schematically illustrates a monitoring system 30 according to the invention. The system is configured for monitoring biological entities while in an incubator 31 (or while the biological entities are at a controlled environment such as temperature and or humidity), and includes such main constructional parts as an optical system 32 and a control unit 38. The optical system 32 is associated with an entity positioning stage 36 located inside the incubator 31 (or in the temperature controlled environment), and the control system 38 is located outside the incubator being connectable to the optical system 32.

To facilitate illustration, all the conventional means of the incubator that may be used with the present invention and that are known per se are not shown in the figure. These means can include inter alia those for controlling temperature, humidity, CO₂ and oxygen levels, light and/or other operating conditions of the incubator. Generally speaking, the incubator 31 is designed and operated to maintain the predetermined environmental conditions. The incubator shelf size is conventionally at least 48×48 cm kept under the following environmental conditions: substantially steady temperature of 37-38° C.; a CO₂ level of 5%, oxygen level between 5 to 25%, relative humidity of about 95%, substantially dark and sterilized (clean) environment. Preferably, the system of the present invention utilizes a novel temperature controlled means developed by the inventors, as will be described more specifically further below. This temperature control means are partly installed within (or associated with) the entity position stage 36 and with elements of the system which may create heat inside the incubator (not shown).

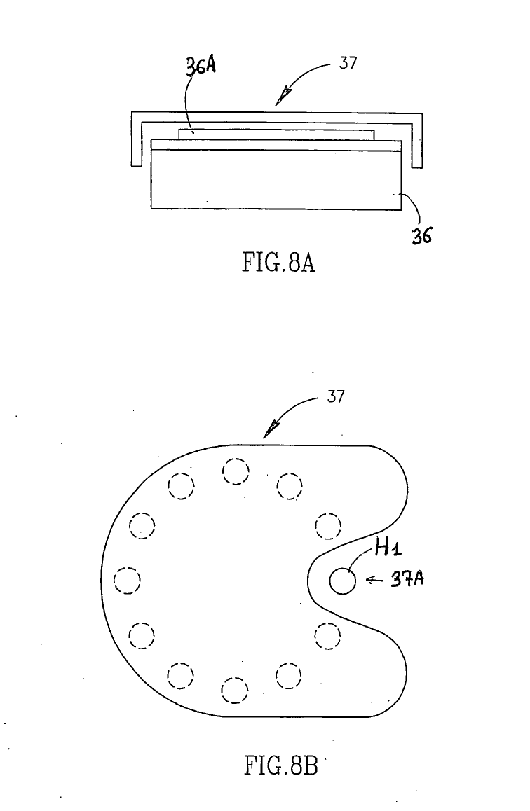

The entity positioning stage 36 includes a support plate 36A designed to define a plurality of N (e.g., 12) sites (e.g., openings) for supporting a plurality of embryos containing holders (e.g., petri dishes) H₁, . . . H_{N}, respectively. It should, however, be noted that generally such a stage can be designed for supporting only one holder, if needed. The optical system 32 comprises illuminating optics, generally at 34, which is appropriately mounted on the stage 36 so as to be slightly above the support plate 36A, and comprises a light detecting arrangement (not shown here) installed inside the stage 36 so as to be below the support plate 36A. It should also be noted that the imaging system may, for example, utilize a camera having a very low light source for illuminating at least a part of the controlled environment, rather then using a separate light source associated with the holder. The stage 36 is preferably designed in accordance with a further aspect of the present invention so as to minimize affecting the biological entities in the incubator (or another temperature controlled environment) caused by unavoidable changes in the incubator environmental conditions associated with opening the incubator, for example to remove thereform a desired sample. To this end, the stage is provided with a specifically designed cover (not shown here), as will be described further below with reference to FIG. 8.

Further provided in the system 30 is a drive assembly 39 that is associated with the support plate (or stage) 36A or illuminating optics 34 or both of them, and is operated by a suitable utility of the control system 38 to bring selected holder(s) to an imaging position. The drive assembly can also be associated with the imaging system so that the imaging system can be moved to imaging position with respect to the holder or the biological entity, with or without moving the supporting stage and/or the illumination means.

The dishes H₁, . . . H_{N} are preferably arranged in a circular array, and the support plate 36A is preferably mounted for rotation with respect to the illuminating optics 34. The rotation of the support plate 36A allows bringing each one of the dishes to an imaging position with respect to the optics 34. The rotation of the plate with dishes is preferably controlled by a phase control system (e.g., associated with the control system 38 or a separate control unit) thus enabling a technician to reach and track each embryo on each and every petri dish upon demand. In this procedure, the imaging system is preferably also moveable to allow a precise positioning thereof and of the biological entity or the holder. The system 30 therefore enables concurrent monitoring and controlling of multiple patients' entities while within the same incubator or incubation environment.

The optical system 32 is an imaging arrangement formed by a single imaging system having variable or fixed magnification or more than one imaging systems of different magnifications. Such an imaging system includes a light source unit, a light directing assembly, and a light detection unit. A light emitting assembly of the light source unit may be located inside the incubator, or outside thereof in which case emitted light is directed towards the inside of the incubator via fiber(s). The light source unit may be of any known kind capable of providing a short exposure of the embryo so as not to cause damage of the latter. Such light sources include visible light, infrared light, ultraviolet light and others. Preferably, the light source utilizes white LED(s). The intensity of light used in a microscope is 600 lumen, which is the equivalent of 35 Watts. In comparison, the white LED light provides 9200 millicandela intensity, which is equivalent to approximately 60 mW.

This correlates with the width of a light beam coming from a 20° direction. These facts make it clear that the intensity of light used in the system of the present invention is lower than light emitted from a microscope and thus it helps maintain the embryos in optimal conditions. The wave length spectrum that is visible to the human eye from light emitted through a microscope varies between 450 nm and 750 nm. This is the same wavelength as is emitted from the white LED light installed in the system of the present invention.

With the conventional approach the monitoring of embryos (i.e., embryos are monitored manually by embryologists, the embryos can be removed from the incubator 1-3 times a day for evaluation, while being exposed to light for approximately 10 minutes a day). The monitoring system of the present invention allows for minimizing the embryos exposure to light as the embryos are only exposed to light for 30 seconds a day (in the case they are photographed 3 times a day).

Prior to placing the optical arrangement in the incubator it must be cleaned or sterilized in order to maintain a clean and/or sterile environment. The system is delivered to the customer's laboratory when it is sealed and ready for use. The monitoring system of the present invention is preferably made of anodized stainless still. Therefore, the following six sterilization steps are to be carried out before delivery to ensure sterility of the device: (1) wipe of the interior elements with 70% ethanol; (2) ultra violet radiation of the device body and the lid separately for an hour; (3) assembly of the lid and the body in a clean environment (laminar flow); (4) wipe again with 70% ethanol; (5) transfer the monitoring system's' optics in a sealed nylon bag to the investigational site; and (6) wipe again with 70% ethanol prior to installing this unit in the investigational incubator.

The system 30 of the present invention is intended for monitoring the entities inside the incubator (generally, under controlled environmental conditions) in a continuous and automatic manner while recording the results by using images and optionally an invariable database of identification marks such as barcodes (reference data) that accompany the process from initiation until the end of the process. This is aimed at evaluating development of the embryos, and eventually selecting the most suitable embryo, within the matching set of a subject (patient), for further implantation in the uterine wall. To this end, the entities, as well as entities' identification codes are imaged.

Generally, the imaging procedure includes the following: simultaneous imaging of the entire dish (all the entity drops in the dish and the ID/identification marks), imaging of the entity drop only, and imaging of the embryo within the drop. These three image acquisition procedures practically need different magnification optics, and therefore can be implemented by three different imaging systems having different image magnification factors and/or fields of view, or by the same imaging system with variable magnification (e.g., changeable lens arrangements). For example, for imaging the entire dish-with-label a magnification factor of 1-2 is used, for imaging the specific entity drop a magnification factor of 2-6 is used, and for imaging the embryo within the entity drop a magnification factor of 10-100 is used.

FIG. 4 schematically illustrates one specific but not-limiting example of the implementation of the imaging system 132 according to the invention. The system 132 comprises a light source assembly 134; a light directing assembly 135 for directing illuminating light from the light source towards the holder H on support plate (36A in FIG. 3) and directing light transmitted through the holder towards a light detector 136 (e.g., color CCD). In the present example, the light source assembly 134 includes a plurality of white LEDs (e.g., 12 LEDs). The light source arrangement may utilize fiber optics. The light directing assembly 135 is configured to enable phase contrast illumination. To this end, the light directing assembly 135 comprises an annular aperture 140 (e.g., having inside and outside dimensions of respectively, 19.7 mm and 60 mm), a light blocking disk-like element 142 of a diameter smaller than the inner diameter of the aperture 140 (e.g., of 16.25 mm diameter), and a first polarizer 144 all located at one side of the support plate; and a lens 146 and a second polarizer 148 located at the other side of he support plate.

The output of the detector is connected to the control system 38. The light blocking disk 142 is mounted for movement along an optical axis of the system 132 with respect to the support plate 36A. This arrangement allows for 3D-imaging of the holder-with-cell.

The optical system 132 preferably includes several CCD cameras 136 which deliver images in various sizes. Starting with a view of the full Petri dish and then to a view of a single embryo the size of the monitor. The illuminating optics (34 in FIG. 3) is placed on a mobile optical table driven for movement in a plane parallel to a plane defined by the support plate (X-Y plane), and preferably also along the Z-axis for auto-focusing purposes to focus all of the lenses (e.g., ×1, ×10, ×20) to a final magnification of ×10, ×100 and ×400.

It should be noted, although not specifically shown, that the monitoring system of the present invention preferably also contains means for carrying out a fertilization stage. Such means may include a zona drilling tool which assists fertilization, or a micromanipulation tool, such as that used in a fertilization process known as intracytoplasmic sperm injection (ICSI). These processes may be automatically operated by displacing (rotating) either the support plate (36A in FIG. 3) or the tool to bring the tool to a respective position with respect to a selected sample. It should, however, be noted that the fertilization may be performed outside the monitoring system. Alternatively it may be done by using a standard entities' mixing procedure, in which case it is preferably carried out automatically: a portion of the sperm entity is automatically brought in contact with an oocyte entity drop. If the fertilization is carried out inside the incubator equipped with the monitoring system of the present invention, the same optical system of the monitoring system may be used for imaging the fertilized oocyte entity and recording the sample-with-IDᵢ image. At times, the fertilized oocyte entity may be assigned with another ID associated with said IDᵢ matching set.

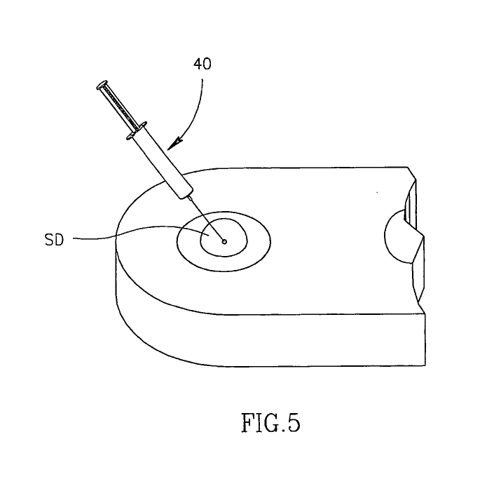

Further preferably provided in the monitoring system of the present invention is a medium changer assembly 40 shown in FIG. 5. The medium changer assembly 40 is aimed at maintaining controlled environment within the entity drop SD (located in a selected site of the dish). The medium changer 40 is typically in the form of a catheter or pipette for adding the required medium into the entity drop such as cleavage medium or blastocyst medium. The medium to be provided to the embryo is typically kept at low temperature, e.g. 4° C. and is warmed (e.g. to a temperature of 37° C.) and gazed before use. The medium changer may also be employed for fertilization or for exposing the embryo to other suitable agents (in example, cryoprotectants or dyes).

Turing back to FIG. 3, the monitoring system 30 of the present invention is designed for continuous use, on a real time basis, from within the incubator. The stage 36 is periodically actuated for rotation, e.g., every three hours, and during the rotation cycle, the entity dishes are successively brought to the imaging position with respect to the camera(s) of the optical system 32. The control system 38 is preferably equipped with a specific electronic card for controlling the movement of the stage so as to bring a specific holder into an imaging position upon demand, in response to data input by an operator. The imaging procedure may be initiated from a remote location via communication with the system 30. The cameras (or a single camera with variable magnification) are operated to acquire images (e.g., three differently magnified images) within the respective dish as described above. For imaging the embryo within the entity drop, the camera is moved with respect to the stationer entity drop. The control system 38 may for example utilize the RS 232 composite video channel or USB protocol/connection for inputting the image related data coming from the camera into a frame grabber card. The magnification factor between the image related data coming from the camera (CCD) and a control system monitor may for example be about 20. The magnification can be carried out by suitable digital means. A 0.1 mm diameter embryo or a 5 mm diameter entity drop can thus be images at the monitor size (54 mm diameter). The control system preferably utilizes specific software enabling simultaneous appearance of all the differently magnified images on the monitor. Preferably, the control system performs the so-called learning mode for each specific set of dishes. The control system can be connectable to other system(s) through any known communication protocol, for example allowing operation of the monitoring and controlling process through the Internet.

It should be noted that the simultaneous imaging of the entire dish-with-label (generally, dish-with-ID) enables real time visualization and evaluation of all the drops within the dish. The evaluation of the embryo stage includes inter alia the following development stages: meiosis, mitosis, number of cells within the embryo (2, 3, . . . 8), blastocyst, etc. The operational cycle extends for up to 7 days per Petri dish, namely, each embryo is kept within the monitoring system unit between 1 to 7 days until obtaining a desired stage in the development of the embryo such as blastocyst ready for implantation.

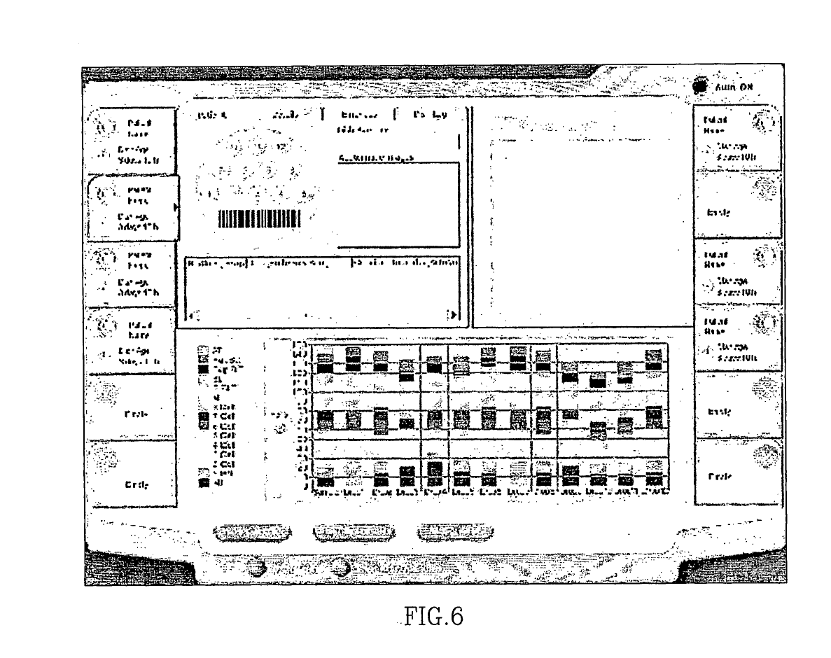

The evaluation scheme is exemplified in FIG. 6. This procedure allows for comparing all the entities simultaneously with a reference entity to thereby improve the quality control of the entity development and selection of the best embryo for implantation. The remaining (non-selected) embryos may then be transferred from the incubator for storage according to any known procedures, e.g., cryopreservation. The technique of the present invention thus enables obtaining and managing of complete documentation relating to the embryo development for each subject.

With computerized assistance, the system can be taught of the location of each embryo by tracking the embryo, centering it on the monitor and saving the location on the invariable database. Saving the data enables work with automatic movement while repeating the cycle for each embryo. The photographs are saved the in the database.

The system that is located within the incubator is exposed to the exact same environmental conditions associated with the incubator itself, including but not limited to the temperature, the air composition and the humidity. The instrument is attached to an external control box that supplies it with a low DC voltage and communication thereof to an electronic card. The control box is affixed to a PC computer and a navigation joystick to the phase control.

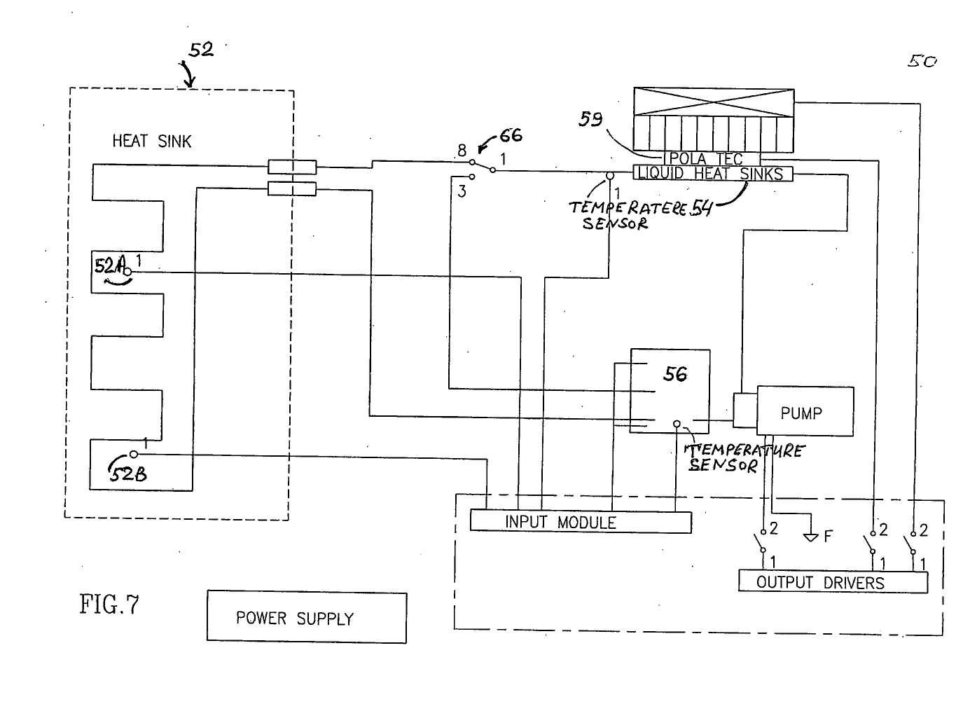

FIG. 7 exemplifies a temperature control system 50 according to the invention suitable to be used in the monitoring system 30. Generally, this temperature control system 50 may advantageously be used in the conventional incubator or any other application for maintaining a required temperature of an object. The system 50 comprises such main constructional parts as a heat sink unit 52 equipped with one or more temperature sensors—two such temperature sensors 52A and 52B being shown in the present example; a second heat sink unit 54 formed by a thermoelectric system 59 and a water reservoir 56 equipped with temperature sensors. Further provided in the control system 50 is a pump assembly 58, suitable input and output modules 60 and 62, and a power supply unit 64. The heat sink units 52 and 54 are connected to each other via a valve (connector) 66. The disconnected (inoperative) position of the valve 66 results in a bypass water circulation mode within the heat sink unit 54. Shifting the valve 66 into its operative position allows water circulation through the heat sink unit 52. The valve is shifted between its inoperative and operative positions depending on the temperature condition in the heat sink unit 52. The input module 60 receives data from all the temperature sensors, thus enabling the operation of water circulation in the system The first heat sink unit 52 with the temperature sensor(s) 52A and 52B is located inside the incubator, being mounted in the stage 36 below the support plate 36A, while all the other parts of the system 50 are located outside the incubator. The second heat sink 54 is preferably an electrical heater and operates to maintain water in the reservoir at certain temperature condition. In this specific example of controlling the temperature condition of embryos to be about 36-37° C. (preferably 36.7° C.), the heat sink 54 operates to maintain the water temperature in the reservoir 56 to be about 32° C.

The system 50 operates in the following manner: Pump assembly 58 operates to provide continuous water circulation in the system, i.e., in the heat sink unit 54 when in the inoperative position of the valve 66 or heat sink unit 52 when in the operative position of the valve. The valve shifting between its inoperative and operative positions is performed in accordance with the temperature condition in the heat sink unit 52, i.e., in the incubator. Upon establishing the required temperature (36-37° C., preferably 36.7° C.) in the heat sink unit 52 and initial water temperature in the reservoir 56 (32° C.) (which is then maintained by the thermoelectric system 59), the valve is maintained normally disconnected (inoperative), and thus no cold water is supplied to the heat sink unit 52, while the temperature condition in the heat sink unit 52, as well as in the reservoir 56 is continuously controlled. Upon detecting that the temperature in the heat sink unit 52 exceeds the predetermined value, the valve is shifted into its operative position and colder water is supplied to the heat sink unit 52 to return its temperature to the required level. If a decrease of the water temperature in the heat sink unit 52 is detected, the thermoelectric system 59 operates to heat water in the reservoir, and the valve 66 is shifted into its operative position.

As indicated above with reference to FIG. 3, the stage 36 supporting a plurality of dishes is preferably provided with a cover according to the invention, that is specifically designed to minimize an effect of change in the incubator environmental conditions (specifically temperature and gas level) on the entities inside the incubator. Such a change is typically caused by opening the incubator to remove thereform a desired biological entity. FIGS. 8A and 8B schematically illustrate side and top views of such a cover 37. As shown, the cover 37 is dimensioned so as to completely cover the entire plate-with-holders, except for a location defined by a recess 37A in the cover 37. The recess is appropriately dimensioned (is slightly larger than a dish H₁). Hence, when access to a specific dish H₁ is required, the plate 36A is rotated (automatically, from outside the incubator via a control system) to locate the specific dish within the recess 37A, while all the other dishes on the plate are covered. The cover 37 may be opaque, in which case it would protect the entities under it also from fluctuations in the lighting environment when the incubator is opened.

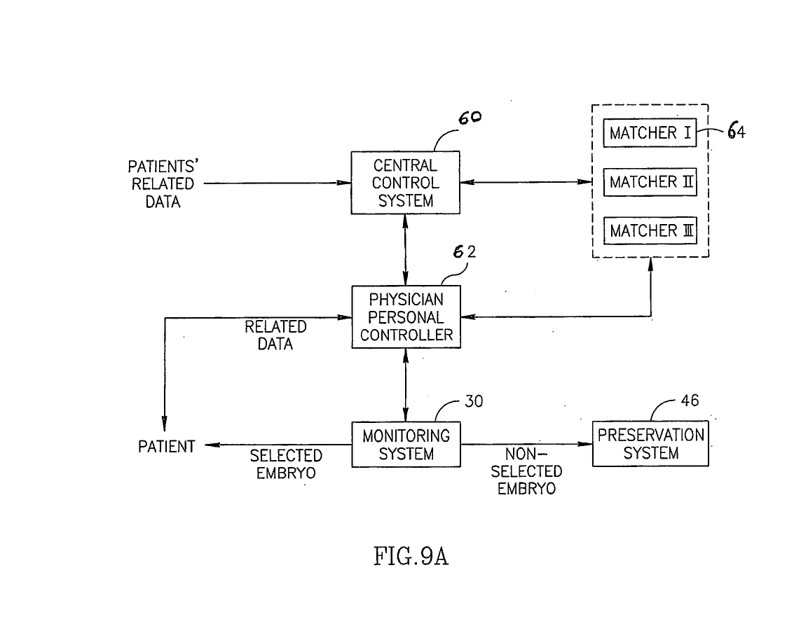

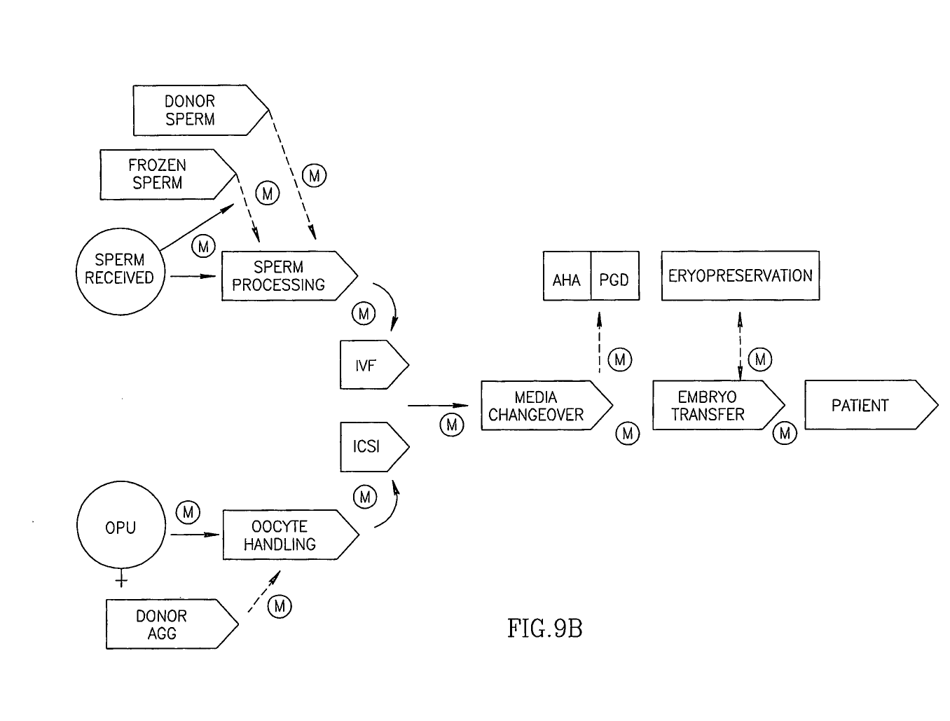

The entire technique of the present invention will now be described more specifically with reference to FIG. 9A. Patients interested in the IVF procedure are assigned with unique IDs, thereby creating matching sets (e.g., pairs) of IDs. Data indicative of the IDs (included in patient's related data) are presented on labels attached to the patients and corresponding entities' containing dishes, and recorded in a central control system 60. The latter is also recorded with data indicative of a physician responsible for a specific procedure. The physician preferably utilizes a personal controller 62 during entities' manipulation, data collection and recording. The physician scans the patient's wrist bracelet with the handheld PC 62, and once a successful match is made between the code on the patient's bracelet and the data located in the memory utility of handheld PC, the procedure is approved. In the event of ovum pick up, a sticker with the patient's barcode identification number is attached to the back of the petri dish that is intended to be placed in the incubator-with-system 30 (constructed and operated as described above with reference to FIG. 3). The handheld PC 62 is returned to the Cradle and connected to the central system 60. The patient's barcode is transmitted to all the system's units and from this point onwards, an external surveillance is conducted for that specific barcode. Handling of the oocytes and conducting the insemination are preformed inside the laminar flow.

Also recorded in the central control system is data indicative of the respective matcher device 64 (one of the plurality of matcher devices) in which matching of entities is verified. Moving the dish H₁ or the test tube H₂ from the matcher unit 64 is immediately detected by optical sensors and transmitted to the central system 60 when matching is completed. The central system 60 transmits the data to the matcher unit 64 that updates the screen with the new status.

After fertilization, the fertilized eggs-containing dish is preferably assigned and labeled with an identification code associated with the specific matching pair. The fertilized eggs-containing dish is then transferred to the incubator-with-system 30 for embryo development, where its development is monitored until selection of the best embryo for implantation. As indicated above, fertilization can be carried out within the system 30. Prior to implantation, the IDs on the selected embryo containing dish and patient are again inspected for matching. Non-selected embryos are transferred to a preservation system 46, and may then be used for future implantation, for example in case of unsuccessful initial implantation or miscarriage.

FIG. 9A illustrates in a self-explanatory manner how the matching procedure of the present invention, generally denoted M, can advantageously be used for controlling the entire IVF procedure. The use of specifically designed identification marks (e.g., barcode) as well as labels with such marks are the basis for accurate identification of elements in the IVF cycle including: culture dishes, test tubes, semen cups, straws, patient bracelets and patient file holders. Each step in the cycle is documented in the system's database. All data recorded in the matcher device can be automatically compiled and stored to produce a variety of reports. As a management procedure, the matching improves OA/OC control without adding complicated procedures. Costly mistakes are avoided and data equivalent to witness verification is efficiently maintained without the involvement of extra personnel.

The following is a specific non-limiting example of the entire technique of the present invention, including the matcher device and the monitoring system and their combined operation.

The system is designed for continuous use, on a real time basis, from within the incubator. The operational cycles extends for up to 7 days per Petri dish. Management of 12 transparent petri dishes, 60 mm in diameter, is conducted via the computer system (formed by the physician's personal computer and central computer station). Each dish is marked with a barcode sticker pursuant to the specifications and the dishes are placed in a single circle on the rotating plate. Identification of the barcode is based upon analysis of the picture via the CCD wide-angled lens camera. The oocyte identification system is comprised of three CCD cameras with the capability of extending to ×10 and ×20 lenses. The cameras are located on the optical table that moves along the three axes. The electric focus equipped with a tiny step motor is placed on the Z-axis. A joystick unit can be used to exercise control over the focus. The background light system providing the cameras is based on the white LED technology. All three cameras are linked to one screen (monitor or PC) using an electronic multiplexer. Control over the system's motors and peripheral components is conducted through a designated electronic card. Control over all the system's operations is exercised through an electronic control board (with an option to control the same via a PC). The connection between the PC and the monitoring system is conducted by appropriate communication, using connection cables or wireless information transmission. The cables are inserted into the incubator through an opening designed specifically for the insertion of standard cables. A composite video line connects the monitoring system to the PC frame grabber card. The system's power suppliers are located in the control box. Processing voltages are preferably as follows: input—80-250 VAC; purveyance—24,12,5 VDC; average current—2A. The system dimensions are 395×470×280 mm. The pictures are stored in a special database on the PC. The database includes all the standard database activities such as, saving, profile extraction, structured search, archive, print etc.

The optical requirements are preferably as follows: CCD at a size of ¼ inch to ⅓ inch; 10-inch window on the computer monitor (based upon the user's choice); CCD resolution of ×10 Camera 450 TV Lines ⅓ inch, X20 Camera 330 TV Lines ¼ inch, and wide-angled camera 330 TV Lines ¼ inch. The increment between the CCD to the monitor is of about 20. During the embryo location scanning, all the dishes appear on the 60 mm diameter screen. During the droplets scanning, a part of a single droplet appears on the entire monitor 1 mm in diameter, the increments is about 80, and the optical increment is about ×10. During the embryo scanning, a single embryo appears on the entire screen at 0.1 mm, over-all increment is of about 400, and the optical increment is of about ×20. Under these increments, each embryo cell that was split into 8 parts will be approximately 40 mm in diameter.

The monitoring system according to the invention is a very useful tool for evaluating the embryo development. It enables to provide the embryos with the optimal conditions without removing them from the incubator, except for performing IVF related procedures. The system offers on line monitoring as well as time laps evaluation of embryos inside the incubator.

The following is the description of experiments conducted by the inventor to prove that the technique of the present invention bears no influence on the development of embryos in a culture dish. The environment in close proximity to the monitoring system, the influence of movement, light and the label toxicity on the embryos have been tested.

Female CB6F1 (3 weeks old) mice bred from stock (obtained from Harlan, Jerusalem, Israel) were kept under controlled conditions (12 h light, 12 h dark) and supplied with water and pellets Teklad Harlan, Israel). Ovaries were stimulated by intraperitoneal injection of 0.1 ml pregnant mare's serum gonadotropin (PMSG) (Sigma, St. louise, USA), and human chorionic gonadotrophin (HCG) (Sigma, St. louise, USA) 47 hours later. The females were mated with FVB/N males. Twelve hours after mating, animals were sacrificed by cervical dislocation and the ampoule was removed immediately into M2 medium (Sigma, St. louise, USA). 2PN stage embryos were flushed into a 300 [symbol] g/ml hyaloronidase (Sigma, St. louise, USA) drop. After washing the embryos three times in M2 and M16 (Sigma, St. louise, USA) they were divided into 20 μl drops, each drop containing 20 embryos and cultured under mineral oil (Sigma, St. louise, USA).

The dishes were placed in the incubator (ThermoForma 3110, Ohio, USA) at 37° C., 5% CO₂ and 95% humidity, equipped with the optical monitoring system of the present invention (an EmbryoGuard™ unit). One control group consisting of 20 embryos in 20 μl drops under mineral oil was placed in the same incubator on another shelf (control 1) and the second control dish (control 2) was placed in another incubator (within which there was no monitoring system of the present invention) with the exact same environmental condition.

The embryo development was evaluated every 24 hours manually. The dishes placed in the EmbryoGuard™ unit, were rotated and pictures were taken. The temperature condition was recorded using a data logger (ALMEMO 2290-4, Germany) connected to a PC.

In the dishes that were used for the sticker experiment, the embryos were divided in the same manner as in the control groups (20 embryos in 20 μl drops under mineral oil). The stickers were attached on the inside upper lid of the petri dish in order to magnify the effect of the glue and ink on the embryos. The dished were placed in the incubator that did not contain the optical monitor.

Embryonic development among different treatment groups were compared initially using Chi-square (Ω²) analysis. In the case significant differences existed among the groups, pair-wise comparisons were made. Probabilities of treatment difference less than 0.05 were considered significant.

The cleavage rate of 2PN embryo in the EmbryoGuard™ 24 hours after fertilization was 82% (115/140), control group 1 was 84% (136/162), control group 2 was 90% (197/220), and the group which tested the toxicity of the stickers was 86% (120/140). The balstocysts formation on the fifth day after fertilization was 45% (52/115), 49% (67/136), 54% (106/197) and 58% (70/120), respectively. Table 1 presents the results of the embryo development in this experiment:

TABLE 1

| | Total | Cleavage rate ± S.E | Blastocyst rate ± S.E |
|---|---|---|---|
| Treatment | 2PN | (N) | (N)*** |
| EmbryoGuard ™ | 140 | 82% ± 3.2 (115) | 45% ± 4.6 (52) |
| *Control 1 | 162 | 84% ± 2.8 (136) | 49% ± 4.2 (67) |
| **Control 2 | 220 | 90% ± 2.0 (197) | 54% ± 3.5 (106) |
| Sticker | 140 | 86% ± 2.9 (120) | 58% ± 4.5 (70) |

Here, indexes *, ** and *** refer to the following:
*Using the same incubator within the EmbryoGuard ™ on another shelf;
**Using another incubator with the exact same environmental condition as that of the EmbryoGuard ™;
***Blastocyte rate out of cleaving embryos.

The results of a previous experiment (Arav et al ICAR 2000) using CCD cameras for continues evaluation of balstocysts and an appropriate background light source inside an incubator, correlate with the above-described experiments which showed normal cleaving development that does not differ from both control groups—one in the same incubator and the other in another with the exact same conditions.

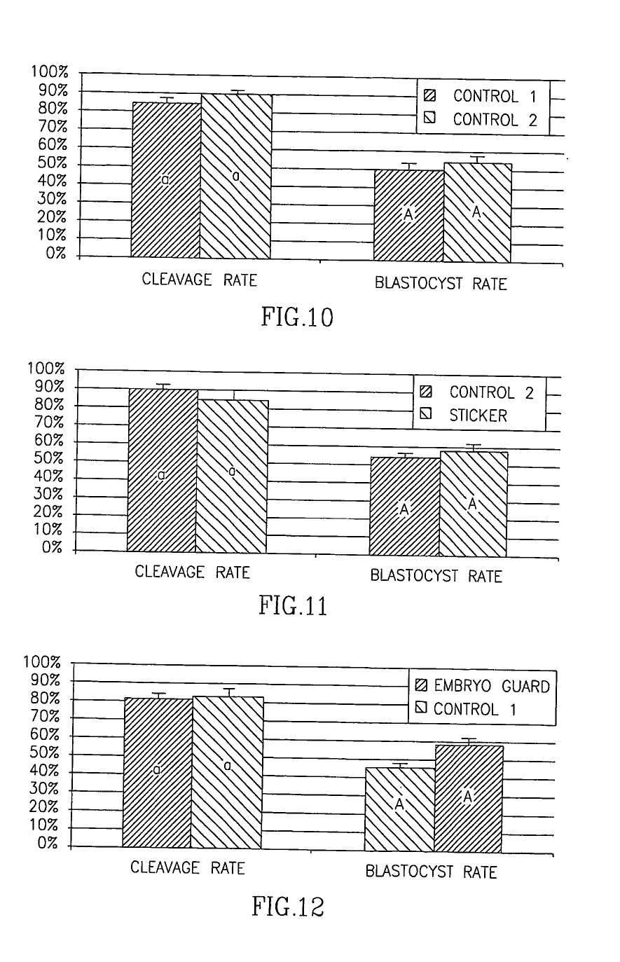

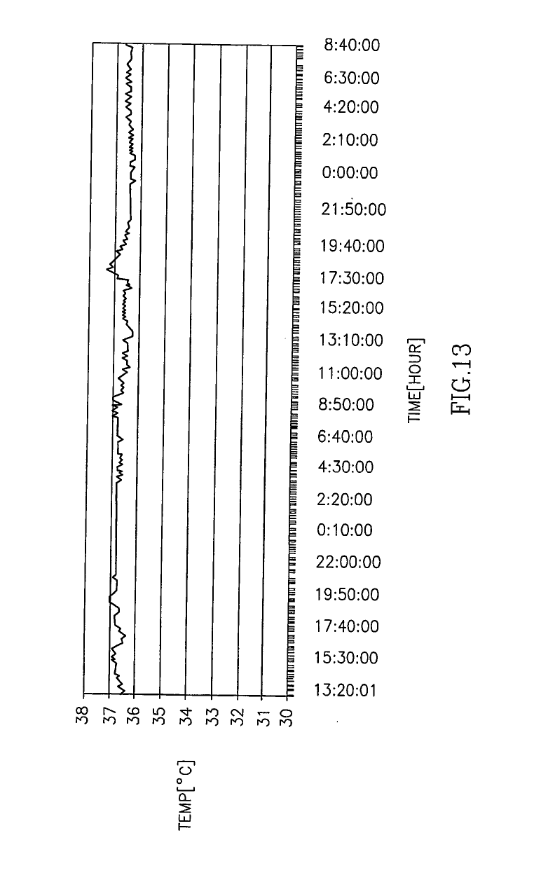

FIG. 10 compares the results obtained for control 1 and control 2 groups. Analysis of these results have shown that there were no significant differences (P<0.05) between the control groups throughout the entire embryonic development. This confirms that the environmental conditions in the incubator with EmbryoGuard™ unit where not disturbed. FIG. 11 compares control 1 group to the EmbryoGuard™ group. This comparison shows that the embryos continued to develop to the blastocyst stage, with no significant differences. FIG. 12 compares control 2 group to the sticker group. The sticker group consistently did not show lower development than control group 2, which incubated in the same incubator. Hence, neither the illumination nor any source of light radiation affects the embryos growth. A suitable incubation environment was maintained while the EmbryoGuard™ operated within the incubator. There was no significant temperature fluctuation and the gas circulation was not impaired, as shown in FIG. 13. The fact that the stainless steel plate rotated during the automatic evaluation process, did not affect the embryos. The labels (glue and ink) were also not found to have a toxic affect on the embryos (as presented in the above Table 1).

Those skilled in the art will readily appreciate that various modifications and changes can be applied to the embodiments of the invention as hereinbefore exemplified without departing from its scope defined in and by the appended claims.

1-74. (canceled)

75. A method for use in controlling the processing of components that are involved in in vitro fertilization (IVF), the method comprising:
defining a matching set of two or more components to be involved in a common IVF process;
assigning each component with a unique machine readable identification mark;
providing on each component the unique machine readable identification mark assigned to said component; and
reading the identification mark on each component, generating data indicative thereof; and analyzing the generated data to determine whether the identification marks of said components belong to said matching set or not.

76. The method of claim 75, wherein two or more components to be involved in the IVF process include at least one component selected from a group consisting of a sperm donor entity, a sperm entity, an ova donor entity, an ova entity, an oocyte entity, a fertilized egg entity, an embryo entity, a recipient entity, holder entities, physicians, technicians and corresponding records or data files.

77. The method of claim 75, wherein said matching set of components includes an embryo entity and a recipient entity.

78. The method of claim 75, wherein said matching set of components includes a sperm entity and an oocyte entity.

79. The method of claim 76, wherein the identification mark is an image readable mark.

80. The method of claim 75, wherein the identification mark is readable by optically scanning the mark.

81. The method of claim 75, wherein the identification mark is barcode.

82. The method of claim 75, wherein said providing of the identification mark comprises attaching to the component a label carrying said identification mark.

83. The method of claim 75, wherein at least one entity is within a holder and said providing of the identification mark comprises attaching to the holder a label carrying said identification mark.

84. The method of claim 75, wherein said providing of the identification mark on the holder comprises printing said identification mark onto the holder.

85. The method of claim 75, wherein said matching set of components includes a biological entity and a corresponding record or file.

86. The method of claim 75, wherein said matching set of components includes a biological entity and a holder.

87. The method of claim 75, wherein the unique machine readable identification mark assigned to each component of a matching set is different.

88. The method of claim 75, wherein the unique machine readable identification marks assigned to the components of a matching set are the same.

89. A system for use in controlling the processing of biological entities, the system comprising:
a support assembly for supporting at least one holder containing biological entities;
an optical device operable to acquire an image of the holder and generating data indicative of at least an identification mark provided on the holder;
a control system connectable to said optical system and operable to actuate the image acquisition and to analyze the data indicative of the acquired images, the control system having a memory utility for storing reference data representative of matching sets of biological entities' associated identification marks, and a processing utility preprogrammed to be response of said data indicative of the acquired images to analyze said data utilizing said reference data and identify whether the biological entities in at least two holders relate to a matching set or not.

90. A system for use in controlling the processing of biological entities, the system comprising:
a supporting stage for supporting at least one holders containing a biological entity;
an incubator unit for maintaining the biological entities under predetermined environmental conditions.
an optical monitoring system operable to acquire images of the entities;
a drive assembly operable to provide a desired relative displacement between said support stage at least a lens arrangement of said optical system, so as to enable bringing a selected holder to an imaging position.
